(19) United States
(12) Patent Application Publication
(10) Pub. No.: US 2013/0261567 A1
(43) Pub. Date: Oct. 3, 2013

(54) Coagulant substance which will be found at the base of a needle to reduce leakage of fluids, mainly blood from around the insertion base of a needle

(71) Applicant: John Lewis Hogan, III, Valdosta, GA (US)

(72) Inventor: John Lewis Hogan, III, Valdosta, GA (US)

(21) Appl. No.: 13/374,754

(22) Filed: Mar. 29, 2012

(51) Int. Cl. A61L 33/00; A61M 5/00; A61L 31/08

(52) U.S. Cl. 604/272

(57) ABSTRACT

My invention is improving the hemodialysis fistula needle as depicted as FIG. 1. In doing this I am replacing a non coagulant type of material used at the base of a needle with a coagulant type substance. This will help to reduce leakage from around the insertion site of the needle. For clarification purposes the needle is connected at the top of the hemodialysis fistula needle and the coagulant substance located at the base of the needle. Once the needle breaks through the skin, it must penetrate until the base of the needle is touching at least the epidermis part of the skin. When this happens, if any blood leaks from around the needle, the coagulant substance coagulates the blood which in turn reduces leakage.

### BRIEF SUMMARY OF THE INVENTION

The advantage of using my invention is because using a coagulant around the base of a needle helps reduce leakage from around the needle after insertion is performed. This coagulant substance helps to improve the dilemma of blood or other fluids from leaking at a higher rate. The shape or color of the coagulant substance is not limited to one shape or one color, so it may be consisting of one or more shape and one or more color.

### DETAILED DESCRIPTION OF THE INVENTION

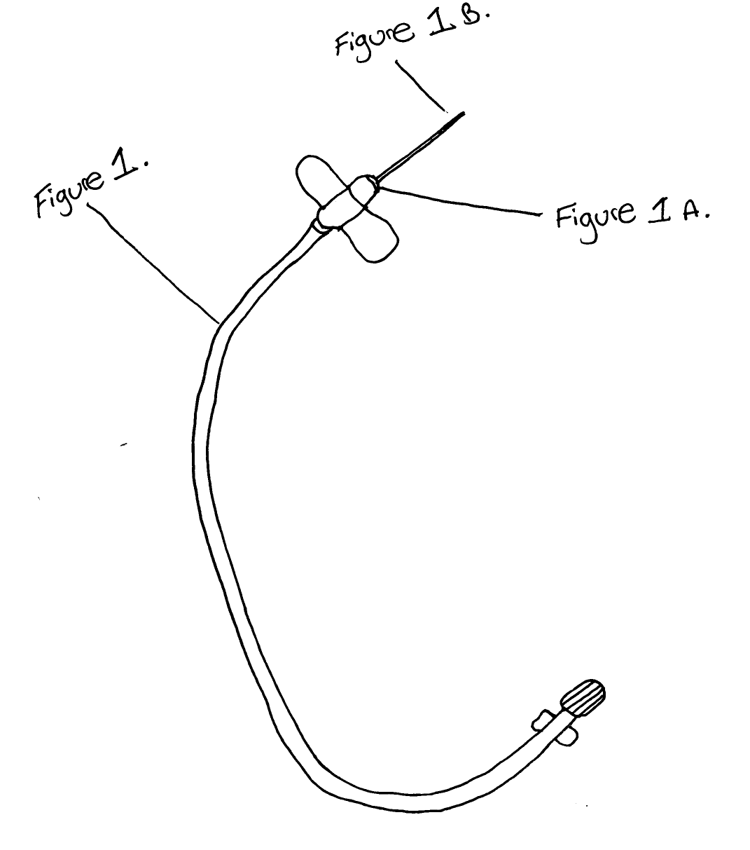

As described in FIG. 1, the coagulant substance is located at the base of a needle. The needle is represented as FIG. 1B in FIG. 1. The coagulant substance is also located in FIG. 1 and is represented as FIG. 1A which is shaded. This coagulant substance is an improvement of non coagulant substances used at the base of needles.

1. A substance for reducing leakage around the base of the insertion site of a needle, said substance is a coagulant used at or around the base of a needle.
